(19) United States
(12) Patent Application Publication
(10) Pub. No.: US 2022/0296130 A1
(43) Pub. Date: Sep. 22, 2022

(54) Sensor Assembly

(71) Applicant: Sanofi, Paris (FR)

(72) Inventors: Michael Helmer, Frankfurt am Main (DE); Maurice Toporek, Frankfurt am Main (DE)

(21) Appl. No.: 17/635,507

(22) PCT Filed: Aug. 25, 2020
(86) PCT No.: PCT/EP2020/073733

§ 371 Date: Feb. 15, 2022

(30) Foreign Application Priority Data
Aug. 26, 2019 (EP) 19306030.8

(51) Int. Cl. A61B 5/11; A61B 5/00

(52) U.S. Cl. CPC A61B 5/1125; A61B 5/6887; A61B 5/4848; A61B 2505/07; A61B 2562/16

(57) ABSTRACT

The present disclosure relates to a sensor assembly including a sensor element configured to be arranged external to a drug delivery device such that a user of the drug delivery device can hold the drug delivery device through gripping the sensor element. The sensor element is configured to detect a contact area of a hand of the user of the drug delivery device when gripping the sensor element. The sensor assembly can be included in a drug delivery system.

### CROSS REFERENCE TO RELATED APPLICATIONS

The present application is the national stage entry of International Patent Application No. PCT/EP2020/073733, filed on Aug. 25, 2020, and claims priority to Application No. EP 19306030.8, filed on Aug. 26, 2019, the disclosures of which are incorporated herein by reference.

### TECHNICAL FIELD

The disclosure relates to a sensor assembly for a drug delivery device. The present disclosure further relates to a drug delivery system and to a system comprising an electronic device, a drug delivery device and a communication link.

### BACKGROUND

Many liquids such as medicaments have to be injected into the body. This applies in particular to medicaments, which are deactivated or have their efficiency remarkably decreased by oral administration, e.g. proteins (such as insulin, growth hormones, interferons), carbohydrates (e.g. heparin), antibodies and the majority of vaccines. Such medicaments are predominantly injected by means of delivery devices such as syringes, medicament pens or medicament pumps.

The users of syringes, medicament pens or medicament pumps can range from healthcare professionals to the medicament-recipient themselves, the latter ranging from children or elderly persons. The medicinal injections may include repetitive or multiple injections of a particular dose (e.g. a vaccine in multi-dosage regimen) to a single injection of a single dose (e.g. a vaccine or in an emergency hydrocortisone).

For this purpose, there are several types of medication delivery devices known such as pen type delivery devices. These delivery devices may be designed as a so called autoinjector. Autoinjectors are medical devices that allow patients to self-administer their medications outside a hospital or physician's office. These devices are often used for the management of chronic diseases, such as diabetes, rheumatoid arthritis, multiple sclerosis, and osteoporosis. A typical autoinjector consists of a prefilled syringe in a mechanical device that deploys a needle and delivers a medicament with a single push of a release button. Other types of autoinjectors do not require the user pushing a release button, but are triggered by a needle sleeve being translated into the device housing. In addition to autoinjectors deploying a needle, there exist also autoinjectors with fixed needles where the user injects the needle into the tissue manually. Still other types of autoinjectors involve a cartridge instead of a prefilled syringe.

### SUMMARY

Despite the advantages provided by autoinjectors, there are still some drawbacks. While autoinjectors allow for convenient self-administration, these devices do not provide feedback to the user, or the user's physician, regarding the state or progression of the disease. Accordingly, prior to using an autoinjector to administer medicine, or during the use of an autoinjector, a patient may need to visit a physician or physical therapist to be given certain tests which can be indicative of the patient's health or the efficacy of the treatment. Based on the results of these tests, the physician may change the course of treatment for the patient.

Currently, drug approval studies for patients suffering from rheumatoid arthritis indicate the effectiveness of a drug with help of questionnaire, e.g. the Rapid or Basdai questionnaire. These questionnaires assess patients' well-being, but not the mobility and/or flexibility of patient joints. The improvement by means of a drug therapy is qualitatively assessed by the attending physician. A quantitative assessment is not performed.

An aspect of the present disclosure can provide a sensor assembly allowing for quantitative assessment of a drug therapy for the treatment of rheumatoid arthritis. Particularly, an implementation can provide a possibility to measure patient's individual change of hand mobility by means of a qualitative measurement method.

In an aspect of the disclosure, there is provided a sensor assembly comprising a sensor element configured to be arranged external to a drug delivery device such that a user of the drug delivery device can hold the drug delivery device through gripping the sensor element, and wherein the sensor element is configured to detect a contact area of a hand of the user of the drug delivery device when gripping the sensor element. Thus, the absolute area of the user's hand contacting the sensor element may be detected. A comparison of different contact operations detected by the sensor assembly allows to determine whether there is a variation or change, e.g. an increase, in the contact area of the user's hand or not.

The sensor element may be touch-sensitive. Alternatively or in addition, the sensor element may be positioned such that there is direct contact between the sensor element and the user's hand, when gripping the sensor element. Alternatively or in addition, the sensor element may be configured to provide the contact area. It has to be noted that the sensor element may be mounted onto an outer surface or into theouter surface of the body of the drug delivery device.

According to a further embodiment, the sensor assembly is configured to be mounted to the outer surface of a body of the drug delivery device. Particularly, the sensor element is configured to be directly or indirectly mounted to the outer surface of a body of a drug delivery device. Thus, the sensor element may be mounted to the outer surface of the body of the drug delivery device without any further element such as an adhesive or may be mounted to the outer surface of the body of the drug delivery device by means of one or more further elements such as an adhesive or a sleeve disposed between the sensor element and the outer surface of the body of the drug delivery device.

According to a further embodiment, the contact area corresponds to the size of an area on the user's hand, where said area on the user's hand touches the sensor element when the user grips the sensor element and/or wherein the contact area corresponds to a position of the contact of the hand of the user of the drug delivery device and/or wherein the contact area corresponds to a number of contacts of the hand of the user of the drug delivery device. The magnitude or extension of the user's capability to grip the sensor area and/or behavior of the user how to grip the sensor area may be detected.

According to a further embodiment, the sensor element is a capacitive sensor element. Alternative sensor types like a polymer thin film resistor sensor element, a thermal sensor element or a resistive sensor element can also be used as sensor element. Such sensor types are well established and allow an exact detection of the contact area. Even small variations of the contact areas with different contacts of the user's hand are detectable.

According to a further embodiment, the sensor element is configured to be wrapped around the outer surface of the body of the drug delivery device. The sensor element may be applied to drug delivery devices different in its outer circumferential dimension.

According to a further embodiment, the sensor element is a flexible label. The sensor element has a broad field of application and may be adjusted to the outer shape of the body of the drug delivery device.

According to a further embodiment, the sensor element is configured to be slipped or clipped over the outer surface of the body of the drug delivery device. The sensor element may be easily mounted to the outer surface of the drug delivery device body by being slipped or clipped over the body. The sensor element may be fixed in its position when slipped or clipped over the outer surface of the body of the drug delivery device such as by means of an adhesive or a moulding process such as foil injection moulding.

According to a further embodiment, the sensor element is an add-on device. Examples for an add-on device are a sleeve or an electronic label. This design further simplifies the mounting of the sensor element. A sleeve may be slipped over the outer surface of the body of the drug delivery device. If the sleeve comprises at least one slit or slot, it may be clipped over the outer surface of the body of the drug delivery device.

According to a further embodiment, the sensor assembly further comprises a sleeve, wherein the sensor element is mounted to the sleeve or is integrated into the sleeve, e.g. a reusable device or add-on.

According to a further embodiment, the sensor element is configured to be integrated into the outer surface of a body of a drug delivery.

According to a further embodiment, the sensor area defined by the sensor element is seized so as to be at least partially contactable by a palm, phalanx and finger joints areas of the user's hand. Thus, usable detection results of the contact area may be obtained with selective areas of a user's hand.

According to a further embodiment, the sensor element is curved. The sensor element may be easily pushed onto the body of the drug delivery device. The curvature of the sensor element is adapted or is adaptable to the curvature of the outer surface of the body of the drug delivery device.

According to a further embodiment, the sensor element is smaller than a total area of the palm phalanx and finger joints areas of the user's hand. There is no need to extend the sensor element to the whole hand but only to a selected surface area thereof.

The palm area serves as reference to detect each individual finger by means of the position, size and course of a palm area. When the patient encompasses the device in a “fully manner”, the sensor element detects the following characteristics of the patient's hand. This sensor element measuring result of a healthy patient or a patient with a very low rheumatism activity. The patient grips the device 100% on the possible sensor area of the sensor element. All fingers and palm delivers the maximum possible measurement value, this depends on the diameter of the outer body or rather the sensor area.

According to a further embodiment, the sensor assembly further comprises an electronic module configured to communicate with an external electronic device.

According to a further embodiment, the electronic module is configured to transmit data representing a detection result of the sensor element to the external electronic device. The detection results obtained by the sensor assembly may be forwarded to an external electronic device in order to be further processed such as for being evaluated. The external electronic device may be located at the drug delivery device or may be a remote electronic device such as a remote computer or the like which is spatially separated from the drug delivery device.

According to a further embodiment, the electronic module comprises a memory configured to store a plurality of detection results of the sensor element. The detection results may be transmitted to the external electronic device at an appropriate point of time.

According to a further embodiment, the electronic module comprises a clock device configured to provide each of the plurality of detection results of the sensor element with a time index indicating the point of time and date of the detection of the sensor element. The detection results may be provided in a timely order. A timely progress of the contact area may be determined.

According to a further embodiment, the electronic module is configured to carry out a comparison of the detection results of the sensor element in a timely order, to carry out an analysis of the comparison and to derive a result of a therapy for the treatment of rheumatoid arthritis from the analysis. Thus, a difference during or rather the change of grip surface may be measured. More grip surface correlates with a better hand mobility assumed that the behavior between the rheumatism severity and the contact surface is linear or approximately linear. The result derived from the analysis may be an improvement or success, a degradation or stagnation of the therapy.

According to a further embodiment, the electronic module comprises a microcontroller configured to control operation of the sensor element, the memory and/or the clock device. The respective components of the sensor assembly may be controlled by a central controller.

According to a further embodiment, the electronic module is mounted to a printed circuit board. The sensor assembly may be miniaturized.

According to a further embodiment, the electronic module comprises a transmitting device configured to transmit the data representing a detection result of the sensor element to the external electronic device in a wireless or wired manner. The signal may be transmitted by means of well established devices decreasing the costs for its manufacturing.

According to a further embodiment, the sensor assembly further comprises a power source configured to power the sensor element. The sensor element may be independent on any external power source.

According to a further embodiment, the sensor assembly further comprises an acceleration sensor configured to detect an acceleration of the drug delivery device or a constructional member of the drug delivery device such as the needle.

According to a further embodiment, the sensor assembly of any preceding claim, further comprising a position sensor configured to detect a position of the drug delivery device. Thus, a relative orientation of the drug delivery device in a three dimensional space may be detected which allows to determine how the use holds the device.

According to a further embodiment, the sensor element is configured to detect an orientation of the user's hand relative to the sensor element or the drug delivery device. The sensor element may detect the user's individual handling of the drug delivery device and learns itself to improve the detection results. For example, the palm area serves as reference to detect the orientation of the hand relative to the sensor element.

According to a further embodiment, the sensor assembly has an ergonomic shape. The sensor assembly may be adapted to patients with rheumatoid arthritis. To that extent, the sensor assembly could be defined to have an ergonomic shape. For example, the sensor element may be provided with recesses and protrusions easing a grabbing of the sensor-assembly-device combination. A simpler feature would involve a sensor assembly having a certain axial diameter e.g. at least 2-3 cm.

In a second aspect of the disclosure, there is provided a drug delivery system, comprising a drug delivery device, and a sensor assembly according to the above described exemplary embodiments, wherein the sensor element is arranged external to the drug delivery device such that a user of the drug delivery device can hold the drug delivery device through gripping the sensor element.

According to a further embodiment, the drug delivery device is an injection device such as an autoinjector. A patient may conduct his or her own test, measurement or data collection of hand mobility by means of the injection device provided with the sensor assembly.

According to a further embodiment, the injection device is configured to inject a medicine for the treatment of a chronic disease, particularly rheumatoid arthritis or other comparable rheumatism diseases. The medicine may be a liquid medicine. A patient may conduct his or her own test, measurement or data collection of hand mobility by means of the injection device provided with the sensor assembly.

According to a further embodiment, the drug delivery device is a mock injection device. A patient may carry out the detection with a dummy device designed similar to a genuine injection device.

According to a further embodiment, the drug delivery device further comprises an electronic device configured to communicate with the sensor assembly. The drug delivery device may directly communicate with the sensor assembly.

According to a further embodiment, the electronic device is configured to inform the user of drug delivery device on the detection result. The use may directly be informed on the detection result so as to learn whether his or her therapy is successful or not.

According to a further embodiment, the electronic device is configured to inform the user of drug delivery device on a variation or change of the contact area from a plurality of detection results. The user may be immediately informed on the progress of his or her therapy.

According to a further embodiment, the electronic device is configured to inform the user of drug delivery device on an increase of the contact area from a plurality of detection results. The user may be rewarded by giving positive information.

According to a further embodiment, the electronic device is configured to inform the user of drug delivery device on the detection result by means of an application running on a mobile communication device such as a smartphone, smartwatch or the like. The information may be displayed by means of a well-established device facilitating the handling of the drug delivery device.

According to a further embodiment, the drug delivery device further comprises a power source configured to supply the sensor assembly with power. The sensor assembly may be powered by the power source of the drug delivery device. Thereby, an own power source for the sensor assembly may be omitted which allows to further miniaturize the sensor assembly.

In a third aspect of the disclosure, there is provided a system comprising an electronic device, a drug delivery device according to the above exemplary embodiments and a communication link between the electronic device and the drug delivery device, wherein the electronic device is configured to receive the detected contact area via the communication link, wherein the electronic device is configured to provide the detected contact area to an output unit.

The communication link may be any realized in a wired or wireless manner. The output unit may be any device that is configured to provide information to the user such as a display unit, a speaker or a database.

The present disclosure further discloses and proposes a computer program including computer-executable instructions for performing the method according to the disclosed method/device/system in one or more of the embodiments enclosed herein when the program is executed on a computer or computer network. The computer program may be stored on a computer-readable data carrier. One, more than one or even all of the method steps as indicated above may be performed by using a computer or a computer network, preferably by using a computer program.

The present disclosure further discloses and proposes a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a computer or computer network, such as into a working memory or main memory of the computer or computer network, may execute the method according to one or more of the embodiments disclosed herein.

The present disclosure further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the present disclosure proposes and discloses a modulated data signal which contains instructions readable by a computer system or computer network, for performing the method according to one or more of the embodiments disclosed herein.

Preferably, referring to the computer-implemented aspects of the disclosure, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present disclosure further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing the findings of the present disclosure, the following embodiments are disclosed:

Embodiment 1: A sensor assembly comprising

a sensor element configured to be arranged external to a drug delivery device such that a user of the drug delivery device can hold the drug delivery device through gripping the sensor element, and

wherein the sensor element is configured to detect a contact area of a hand of the user when gripping the sensor element.

Embodiment 2: The sensor assembly of the preceding embodiment, wherein the sensor assembly is configured to be mounted to an outer surface of a body of the drug delivery device.

Embodiment 3: The sensor assembly of any preceding embodiment, wherein the contact area corresponds to the size of an area on the user's hand, where said area on the user's hand touches the sensor element when the user grips the sensor element and/or wherein the contact area corresponds to a position of the contact of the hand of the user of the drug delivery device and/or wherein the contact area corresponds to a number of contacts of the hand of the user of the drug delivery device.

Embodiment 4: The sensor assembly of any preceding embodiment, wherein the sensor element is a capacitive sensor element, a polymer thin film resistor sensor element, a thermal sensor element or a resistive sensor element.

Embodiment 5: The sensor assembly of any preceding embodiment, wherein the sensor element is configured to be wrapped around the outer surface of the drug delivery device.

Embodiment 6: The sensor assembly of the preceding embodiment, wherein the sensor element is a flexible label.

Embodiment 7: The sensor assembly of any one of embodiments 1 to 4, wherein the sensor element is configured to be slipped or clipped over the outer surface of the body of the drug delivery device.

Embodiment 8: The sensor assembly of embodiment 7, wherein the sensor element is a sleeve.

Embodiment 9: The sensor assembly of any one of embodiments 1 to 4, further comprising a sleeve configured to be disposed at the outer surface of a body of a drug delivery, wherein the sensor element is mounted to the sleeve or is integrated into the sleeve.

Embodiment 10: The sensor assembly of any one of embodiments 1 to 4, wherein the sensor element is configured to be integrated into the outer surface of a body of a drug delivery device.

Embodiment 11: The sensor assembly of any preceding embodiment, wherein the sensor element is seized so as to be at least partially contactable by a palm, phalanx and finger joints areas of the user's hand.

Embodiment 12: The sensor assembly of any preceding embodiment, wherein the sensor element is curved.

Embodiment 13: The sensor assembly of the preceding embodiment, wherein the sensor element is smaller than a total area of the palm, phalanx and finger joints areas of the user's hand.

Embodiment 14: The sensor assembly of any preceding embodiment, further comprising an electronic module configured to communicate with an external electronic device

Embodiment 15: The sensor assembly of the preceding embodiment, wherein the electronic module is configured to transmit data representing a detection result of the sensor element to the external electronic device.

Embodiment 16: The sensor assembly of the preceding embodiment, wherein the electronic module comprises a memory configured to store a plurality of detection results of the sensor element.

Embodiment 17: The sensor assembly of the preceding embodiment, wherein the electronic module comprises a clock device configured to provide each of the plurality of detection results of the sensor element with a time index indicating the point of time and date of the detection of the sensor element.

Embodiment 18: The sensor assembly of the preceding embodiment, wherein the electronic module is configured to carry out a comparison of the detection results of the sensor element in a timely order, to carry out an analysis of the comparison and to derive a result of a therapy for the treatment of rheumatoid arthritis from the analysis.

Embodiment 19: The sensor assembly of the preceding embodiment, wherein the electronic module comprises a microcontroller configured to control operation of the sensor element, the memory and/or the clock device.

Embodiment 20: The sensor assembly of any one of embodiments 14 to 19, wherein the electronic module is mounted to a printed circuit board.

Embodiment 21: The sensor assembly of any one of embodiments 14 to 20, wherein the electronic module comprises a transmitting device configured to transmit the data representing a detection result of the sensor element to the external electronic device in a wireless or wired manner.

Embodiment 22: The sensor assembly of any preceding embodiment, further comprising a power source configured to power the sensor element.

Embodiment 23: The sensor assembly of any preceding embodiment, further comprising an acceleration sensor configured to detect an acceleration of the drug delivery device or a constructional member of the drug delivery device.

Embodiment 24: The sensor assembly of any preceding embodiment, further comprising a position sensor configured to detect a position of the drug delivery device.

Embodiment 25: The sensor assembly of any preceding embodiment, wherein the sensor element is configured to detect an orientation of the user's hand relative to the sensor element or the drug delivery device.

Embodiment 26: The sensor assembly of the preceding embodiment, wherein the sensor element is configured to detect the orientation of the user's hand based on a palm area serving as reference.

Embodiment 27: The sensor assembly of any preceding embodiment, wherein the sensor assembly comprises an ergonomic shape.

Embodiment 28: A drug delivery system, comprising

a drug delivery device, and

a sensor assembly according to any preceding embodiment,

wherein the sensor element is arranged external to the drug delivery device such that a user of the drug delivery device can hold the drug delivery device through gripping the sensor element.

Embodiment 29: The drug delivery system according to embodiment 28, wherein the drug delivery device is an injection device.

Embodiment 30: The drug delivery device according to embodiment 29, wherein the injection device is configured to inject a medicine for the treatment of a chronic disease, particularly rheumatoid arthritis.

Embodiment 31: The drug delivery device according to embodiment 28, wherein the drug delivery device is a mock injection device.

Embodiment 32: The drug delivery device according to any one of embodiments 28 to 31, further comprising an electronic device configured to communicate with the sensor assembly.

Embodiment 33: The drug delivery device according to embodiment 32, wherein the electronic device is configured to inform the user of drug delivery device on the detection result.

Embodiment 34: The drug delivery device according to embodiment 32 or 33, wherein the electronic device is configured to inform the user of drug delivery device on a variation or change of the contact area from a plurality of detection results.

Embodiment 35: The drug delivery device according to any one of embodiments 32 to 34, wherein the electronic device is configured to inform the user of drug delivery device on the detection result by means of an application running on a mobile communication device.

Embodiment 36: The drug delivery device according to any one of embodiments 28 to 35, further comprising a power source configured to supply the sensor assembly with power.

Embodiment 37: A system comprising

an electronic device,

a drug delivery device according to any one of embodiments 28 to 36, and

a communication link between the electronic device and the drug delivery device, wherein the electronic device is configured to receive the detected contact area via the communication link,

wherein the electronic device is configured to provide the detected contact area to an output unit.

Embodiment 38: A system comprising

an electronic device and

a sensor assembly according to any one of embodiments 1 to 27, wherein the sensor assembly is configured to communicate with the electronic device.

Embodiment 39: A computer program, particularly an application, including computer-executable instructions for performing the functions of the sensor assembly according to any one of embodiments 1 to 27 when the program is executed on a computer or computer network.

Embodiment 40: A feedback module, particularly a screen, configured to provide a user with at least one of the detection results from the sensor assembly according to any one of embodiments 1 to 27.

Embodiment 41: A method of mounting the sensor assembly according to any one of embodiments 1 to 27 to a drug delivery device, wherein the method comprises arranging the sensor element external to the drug delivery device such that a user of the drug delivery device can hold the drug delivery device through gripping the sensor element, wherein arranging the sensor element includes:

(a) slipping or clipping the sensor element over an outer surface of a body of the drug delivery device or

(b) providing a sleeve and mounting the sensor element to the sleeve or integrating the sensor element into the sleeve and slipping or clipping the sleeve over an outer surface of a body of the drug delivery device, or

(c) integrating the sensor element into an outer surface of a body of the drug delivery.

It shall be understood that the sensor assembly of claim 1, the drug delivery system of claim 13 and the system of claim 15 have similar and/or identical preferred embodiments as defined in the dependent claims.

It shall be understood that an embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE FIGURES

In the following drawings:

FIG. 1 shows schematically and exemplarily an embodiment of a sensor assembly,

FIG. 2 shows a sensor area defined by the sensor element projected onto a hand of a user of the drug delivery device,

FIG. 3 shows a sensor area unwrapped from the drug delivery device,

FIG. 4 shows a contact area within the sensor area projected onto a hand of a user of the drug delivery device,

FIG. 5 shows an orientation of fingers relative to the sensor area, and

FIG. 6 shows constructional members of an electronic module of the sensor assembly.

### DETAILED DESCRIPTION

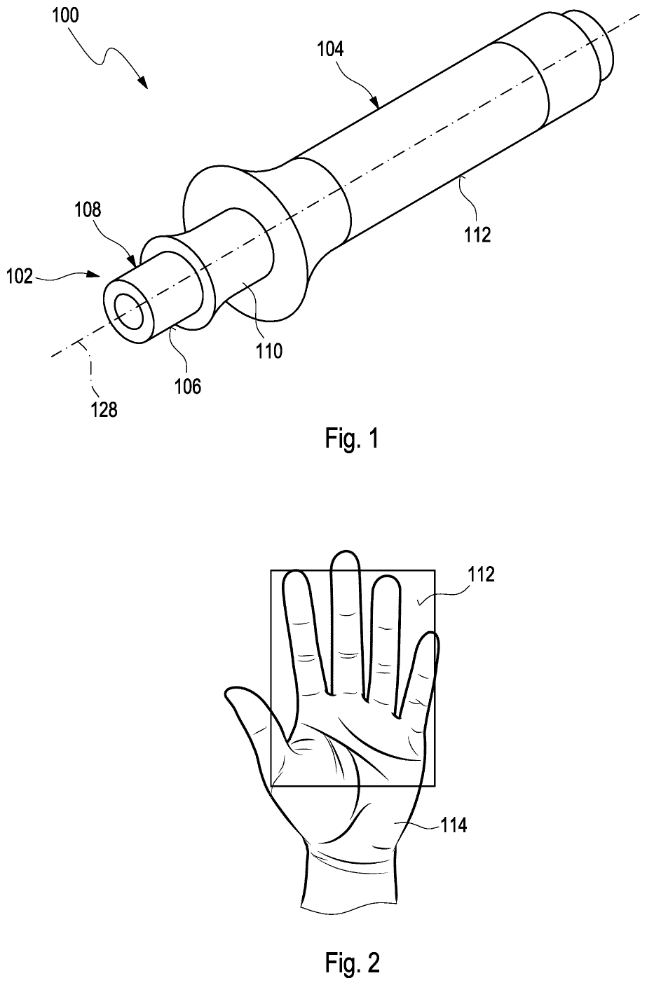

FIG. 1 shows schematically an example of an embodiment of a sensor assembly 100. As will be explained in further detail below, the sensor assembly 100 is configured to be mounted to a drug delivery device 102. The sensor assembly 100 is specifically designed for use in cooperation with an autoinjector, but it can also be used with a mock drug delivery device. Particularly, the drug delivery device is an injection pen type drug delivery device.

As used herein, the term “drug delivery device” relates to a genuine drug delivery device as well as dummy or mock drug delivery device unless otherwise stated.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Determinations like measuring, et cetera performed by one or several units or devices can be performed by any other number of units or devices. For example, detecting can be performed by a single unit of by any other number of different units. The determinations and/or the control of the system for use in accordance with the disclosed method for manufacturing the drug delivery device or administration of a dose can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems. The term “computer program” may also refer to embedded software.

Any reference signs in the claims should not be construed as limiting the scope.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

The sensor assembly 100 comprises a sensor element 104. The sensor element 104 is configured to be arranged external to the drug delivery device 102 such that a user of the drug delivery device 102 can hold the drug delivery device 102 through gripping the sensor element 104, The sensor element 104 is configured to detect a contact area of a hand of the user of the drug delivery device 102 when gripping the sensor element 104. Particularly, the sensor element 104 is configured to be mounted to or disposed at an outer surface 106 of a body 108 of the drug delivery device 102. More particularly, the sensor element 104 is configured to be directly or indirectly mounted onto or into an outer surface 106 of a body 108 of the drug delivery device 102. The body 108 is substantially cylindrical. In the embodiment 100 shown, the sensor assembly 100 further comprises a sleeve 110. The sleeve 110 is substantially cylindrical. The sensor element 104 is mounted to the sleeve 110. The sensor element 104 is curved. The sensor element 104 may be slipped over the outer surface 106 of the body 108 of the drug delivery device 102 by slipping the sleeve 110 over the body 108. The sensor element 104 is a capacitive sensor element. The sensor assembly 100 further comprises a sensor area 112 defined by the sensor element 104.

FIG. 2 shows the sensor area 112 defined by the sensor element 104 projected onto a hand 114 of a user of the drug delivery device 102. As shown in FIG. 2, the sensor area 112 and the sensor element 104, respectively, is smaller than a total area of the hand 114.

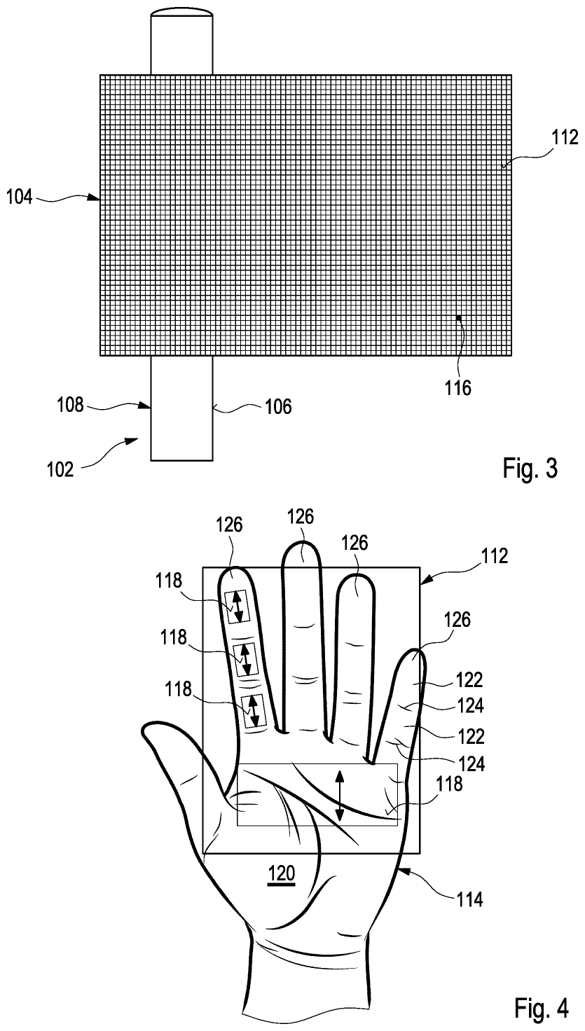

FIG. 3 shows the sensor area 112 unwrapped from the drug delivery device 102. As can be taken from FIG. 3, the sensor area 112 circumferentially covers the outer surface 106 of the body 108 of the drug delivery device. The sensor area 112 may be defined by a plurality of pixels 116.

FIG. 4 shows a contact area 118 within the sensor area 112 projected onto the hand 114 of the user of the drug delivery device 102. The sensor element 104 is configured to detect the contact area 118 of the hand 114 of the user of the drug delivery device 102 when gripping the drug delivery device 102 within the sensor area 112 of the sensor element 104. The contact area 118 corresponds to the size of an area on the user's hand 114, where said area on the user's hand 114 touches the sensor element 104 when the user grips the sensor element 104. Alternatively or in addition, the contact area 118 corresponds to a position of the contact of the hand 114 of the user of the drug delivery device 102. Alternatively or in addition, the contact area corresponds to a number of contacts of the hand 114 of the user of the drug delivery device 102. The contact area 118 includes a magnitude or size of a contact of the hand 114 of the user of the drug delivery device 102 and/or a position of the contact of the hand 114 of the user of the drug delivery device 102 and/or a number of contacts of the hand 114 of the user of the drug delivery device 102. The number of contacts relates to the number of different contact sub-areas of the hand 114 of the user of the drug delivery device 102 at the same time. The sensor area 112 and the sensor element 104, respectively, is seized so as to be at least partially contactable by a palm 120, phalanx 122, and finger joints areas 124 of the user's hand 114. Particularly, the sensor area 112 is smaller than a total area of the palm 120, phalanx 122 and finger joints areas 124 of the user's hand 114. As exemplarily shown in FIG. 4, the contact area 118 may have a variable size as the contact by the palm 120, the phalanges 122 and finger joint areas 124 may vary.

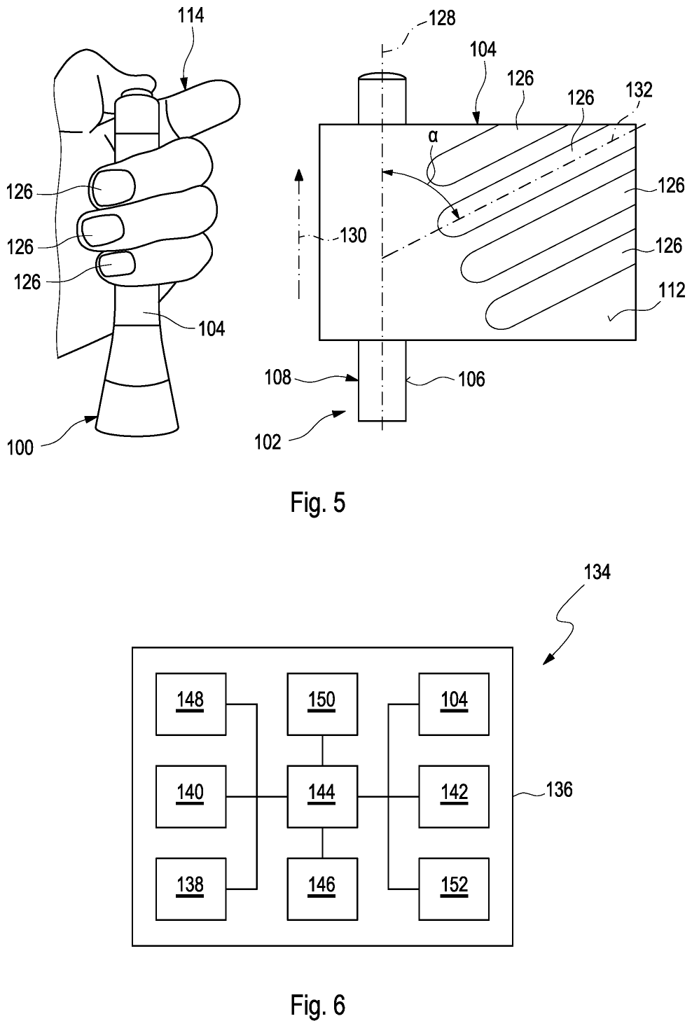

FIG. 5 shows an exemplary orientation of fingers 126 relative to the sensor area 112. Particularly, the sensor element 104 is configured to detect an orientation of the user's hand 114 within the sensor area 112. As shown in the left portion of FIG. 5, the user may grip the drug delivery device 102 with his or her fingers 126 not exactly perpendicular to a longitudinal axis 128 of the drug delivery device 102 but inclined thereto. As such, as shown in the right portion of FIG. 5, the orientation of the fingers 126 relative to the sensor area 112 may be defined as an angle a between an axis 130 of the sensor area 112 parallel to the longitudinal axis 128 of the drug delivery device 102 and a finger extension axis 132. The sensor element 104 may also detect an orientation of the palm 120 relative to the sensor area 112. Particularly, the palm area serves as reference to detect relative orientation of the hand 114 to the sensor element 104.

The sensor assembly may further comprise an electronic module 134. FIG. 6 shows constructional members of the optional electronic module 134 of the sensor assembly 100. The electronic module 134 is mounted to a printed circuit board 136. The electronic module 134 is configured to communicate with an external electronic device (not shown in detail). For this purpose, a communication link between the electronic device and the drug delivery device may be present, wherein the electronic device is configured to receive the detected contact area 118 via the communication link. The communication link may be realized in a wireless or wired manner. The external electronic device may be part of the drug delivery device 102 or may be a remote electronic device spatially separated from the drug delivery device 102 such as a remote computer or a mobile communication device such as a smartphone or the like. In this respect, the term “remote electronic device” refers to an electronic device that is not directly adjacent to the drug delivery device 102 and the sensor assembly 100, respectively, but is connected thereto or communicates therewith. The remote electronic device is spatially separated from the drug delivery device 102 and the sensor assembly 100, respectively, so as to be a separate device. The detection results may be remotely displayed or processed on a separate device.

The electronic module is configured to transmit data representing a detection result of the sensor element 104 to the external electronic device. For this purpose, the electronic module 134 comprises a transmitting device 138 configured to transmit the data representing a detection result of the sensor element 104 to the external electronic device in a wireless or wired manner. For example, the transmitting device 138 is a Bluetooth device. The electronic device is configured to provide the detection detected contact area 118 to an output unit such as a display unit, a speaker or a database. The electronic module 134 comprises a memory 140 configured to store a plurality of detection results of the sensor element 104. The electronic module 134 comprises a clock device 142 configured to provide each of the plurality of detection results of the sensor element 104 with a time index indicating the point of time and date of the detection of the sensor element 104. The electronic module 134 is configured to carry out a comparison of the detection results of the sensor element 104 in a timely order, to carry out an analysis of the comparison and to derive a result of a therapy for the treatment of rheumatoid arthritis from the analysis. The electronic module 134 comprises a microcontroller 144 configured to control operation of the sensor element 104, the memory 140 and/or the clock device 142. The sensor assembly 100 may further comprise a power source 146 configured to power the sensor element 104. The power source 146 may be a battery of the electronic module 134. The electronic module 134 may optionally further comprise an acceleration sensor 148 configured to detect an injection operation of the drug delivery device 102. For example, the acceleration sensor 148 may detect an acceleration of the needle of the autoinjector. If the detected acceleration of the needle is greater than a threshold, then it can be determined that the needle has been operated correctly. The sensor assembly 100 may optionally further comprise a position sensor 150 configured to detect a position of the needle of the autoinjector. The position sensor 150 may be a separate sensor or may be part of the sensor element 104. Thereby, the insertion position of the needle may be detected which correlates with the insertion depth. Alternatively or in addition, the orientation of the drug delivery device within a three dimensional space may be detected.

The sensor assembly 100 may optionally further comprise a structure borne sound sensor 152 as described exemplary in WO 2009/068345 A2.

Referring back to FIG. 1, the drug delivery device 102 is an injection device such as an autoinjector. Particularly, the injection device is configured to inject a liquid medicine for the treatment of a chronic disease, particularly rheumatoid arthritis. As mentioned above, alternatively, the drug delivery device is a mock injection device. The drug delivery device 102 may comprise an electronic device configured to communicate with the sensor assembly 100. The electronic device is configured to inform the user of drug delivery device 102 on the detection result. Particularly, the electronic device is configured to inform the user of drug delivery device on a variation or change such as an increase of the contact area from a plurality of detection results. For example, the electronic device is configured to inform the user of drug delivery device 102 on the detection result by means of an application running on a mobile communication device such as a smartphone. As mentioned above, the drug delivery device 102 may comprise a power source configured to supply the sensor assembly 100 with power.

Hereinafter, an exemplary operation of the sensor assembly 100 is described. When the sensor assembly 100 is mounted to the drug delivery device 102 and supplied with power from the power source 146, the user grips the drug delivery device 102. Thereby, the user's hand 114 partially contacts the sensor area 112. In this respect, it has to be noted that the drug delivery device 102 may be a pen type drug delivery device or mock pen type drug delivery device such that the handling and orientation thereof is like with a pen type injector device. The contact area 118 within the sensor area 112 from the palm 120, phalanges and/or finger joint elements 124 is detected. More particularly, the size of the contact area 118 within the sensor area 112 from the palm 120, phalanges and/or finger joint elements 124 is detected. This detection result is provided with the time index indicating the point of time and date of the detection of the sensor element 104. Further the detection result is stored with the memory 140. This detection operation is repeated each time the user grips the drug delivery device 102 at the sensor area 112. For example, the sizes of the contact area 118 from different gripping operations are detected and timely ordered so as to provide a plurality of detection results. The respective sizes of the thus obtained detection results are compared and subsequently analyzed. If the sizes of the contact areas 118 in the timely order reveal an increase, then an improvement or success of the therapy may be derived as the comparison indicates that the user shows the capability to grip the sensor element 104 to a larger extension. The detection results are transmitted to the electronic device. The electronic device may inform the user of the drug delivery device 102 on the detection result by means of an application running on a mobile communication device such as a smartphone. With other words, when the patient encompasses the drug delivery device during injection, the sensor assembly measures the patients hand contact surface to the sensor area. When the patient hand contact surface grows on the sensor area, the rheumatism activity decreases. The sensor assembly 100 realizes slightest shift and rewards the patient by means of the connected electronic device. Alternatively, the electronic device is a remote electronic device such as a computer spatially separated from the drug delivery device, which may also fulfil all of the functions of the electronic device such as informing a person on the detection result such as by means of a display.

To measure an improvement of the joints mobility, it is not necessary to measure the complete hand grasp area. The electronic system of the sensor assembly can detect repeatedly the patients individual fingers. To recognize an improvement of rheumatism activity it is sufficient to have a measurement of one individual finger pixel segment area. The improvement is measurable, when more finger segments come along. This corresponds to a lower rheumatism activity. By increase of the measurable finger segment, the electronic rewards the patient. Later on, when the patient is able to encompass the device in a fully manner. The sensor element 104 can increase the resolution by reduction of the electronic interpolation to measure the pure grasp surface area of each palm or finger segment. The sensor assembly is self-learning and can after unify uses repeatable recognize each finger (index finger, middle finger, ring finger and little finger). In detail, each bigger measurable surface corresponds with a decreasing rheumatism activity. For example, when the electronic device notices an improvement of haptic properties, the app rewards the patient, e.g. by displaying “Congratulations, did you notice that you can better grasp?”. To encourage patients can be a huge benefit for the patient and as also for the drug maker when an electronic tool can help to reduce the drop-out rate. This disclosure describes a sensor assembly on the drug delivery device combined with an electronic device to reward patients when the drug treatment is successful.

Particularly, the electronic device is the device evaluating the detection results of the sensor assembly. The electronic device processes data acquired from the sensor assembly, particularly data concerning the orientation of contact areas of the user's hand relative to the sensor area, the size of the contact areas and the number of contact areas. A variation or change such as an increase of the size of the contact areas from the respective detection operations and/or an increase of the number of contact sub-areas of the user's hand may be qualified as a success of a patient's therapy and be displayed to the patient. This qualification is based on the assumption that an increase of a total contact area relative to a reference value of a predetermined value such as an increase of at least 5%, 10% or the like represents an improvement of the therapy. Alternatively, a statistically relevant increase relative to a reference value represents an improvement of the therapy. The increase is related to a reference value. The reference value may be the last detected value of the contact area, the first detected value of the contact area or an average value of the last two, three, four, five or the like detected values. Alternatively, an increase in the number of contact areas may be qualified as an improvement of the therapy.

The sensor assembly 100 may be modified as follows within the scope of the present invention. The sensor element 104 may be a polymer thin film resistor sensor element, a thermal sensor element or a resistive sensor element. The sensor element 104 may be configured to be directly mounted to the outer surface 106 of the body 108 of the drug delivery device 102. Particularly, the sensor element 104 may be configured to be wrapped around the outer surface 106 of the body 108 of the drug delivery device 102. For example, the sensor element 104 may be a flexible label. As another modification within the scope of the present invention, the sensor element 104 may be a sleeve. As a further modification within the scope of the present invention, the sensor element 104 may be configured to be integrated into the outer surface 106 of a body 108 of a drug delivery device 102. As a further modification within the scope of the present invention, the sensor element 104 may be clipped over outer surface 106 of the body 108 of the drug delivery device 102. For example, the sensor assembly 100 may be mounted to a slotted sleeve which may be clipped over the outer surface 106 of the body 108 of the drug delivery device 102. Alternatively, the sensor element 104 may be integrated into the sleeve 110. Alternatively, the sensor assembly 100 may comprise an ergonomic shape. The sensor assembly 100 may be adapted to patients with rheumatoid arthritis. To that extent, the sensor assembly 100 could be defined to have an ergonomic shape. For example, the sensor element 104 may be provided with recesses and protrusions easing a grabbing of the sensor-assembly-device combination. A simpler feature would involve a sensor assembly having a certain axial diameter e.g. at least 2-3 cm.

### LIST OF REFERENCE NUMBERS

100 sensor assembly
102 drug delivery device
104 sensor element
106 outer surface
108 body
110 sleeve
112 sensor area
114 hand
116 pixel
118 contact area
120 palm
122 phalanx
124 finger joint area
126 finger
128 longitudinal axis
130 axis
132 finger extension axis
134 electronic module
136 printed circuit board
138 transmitting device
140 memory
142 clock device
144 microcontroller
146 power source
148 acceleration sensor
150 position sensor
152 structure borne sound sensor
α angle

1.-15. (canceled)

16. A sensor assembly comprising:
a sensor element configured to be arranged external to a drug delivery device that is configured to be held by a user of the drug delivery device through gripping of the sensor element,
wherein the sensor element is configured to detect a contact area of a hand of the user when the user grips the sensor element.

17. The sensor assembly of claim 16, wherein the sensor assembly is configured to be mounted to an outer surface of a body of the drug delivery device.

18. The sensor assembly of claim 16, wherein the contact area corresponds to a size of an area on the user's hand, wherein the area on the user's hand touches the sensor element when the user grips the sensor element and/or wherein the contact area corresponds to a position of the contact of the hand of the user of the drug delivery device and/or wherein the contact area corresponds to a number of contacts of the hand of the user of the drug delivery device.

19. The sensor assembly of claim 16, wherein:
the sensor element is configured to be wrapped around the outer surface of the body of the drug delivery device, or
the sensor element is configured to be slipped or clipped over the outer surface of the body of the drug delivery device or
the sensor assembly further comprises a sleeve configured to be disposed at the outer surface of the body of the drug delivery device, wherein the sensor element is mounted to the sleeve or is integrated into the sleeve, or
the sensor element is configured to be integrated into the outer surface of the body (108) of the drug delivery device.

20. The sensor assembly of claim 16, wherein the sensor element is sized so as to be at least partially contactable by a palm, phalanx, and finger joints areas of the user's hand.

21. The sensor assembly of claim 20, wherein the sensor element is smaller than a total area of the palm, phalanx and finger joints areas of the user's hand.

22. The sensor assembly of claim 16, further comprising an electronic module configured to communicate with an external electronic device

23. The sensor assembly of claim 22, wherein the electronic module is configured to transmit data representing a detection result of the sensor element to the external electronic device.

24. The sensor assembly of claim 22, wherein the electronic module comprises a memory configured to store a plurality of detection results of the sensor element.

25. The sensor assembly of claim 16, wherein the electronic module comprises a clock device configured to provide each of the plurality of detection results of the sensor element with a time index indicating the point of time and date of the detection of the sensor element, wherein the electronic module is configured to carry out a comparison of the detection results of the sensor element in a timely order, to carry out an analysis of the comparison and to derive a result of a therapy for the treatment of rheumatoid arthritis from the analysis.

26. The sensor assembly of claim 16, wherein the sensor element is configured to detect an orientation of the user's hand relative to the sensor element or the drug delivery device.

27. The sensor assembly of claim 16, wherein the sensor assembly comprises an ergonomic shape.

28. A drug delivery system, comprising:
a drug delivery device, and
a sensor assembly comprising a sensor element,
wherein the sensor element is configured to be arranged external to the drug delivery device , and wherein the drug delivery device is configured to be held by a user of the drug delivery device through gripping of the sensor element.

29. The drug delivery system of claim 28, wherein the drug delivery device is an injection device configured to inject a medicine for the treatment of a chronic disease.

30. The drug delivery system of claim 29, wherein the chronic disease comprises rheumatoid arthritis.

31. The drug delivery system of claim 28, wherein the sensor assembly is configured to be mounted to an outer surface of a body of the drug delivery device.

32. A system comprising:
an electronic device;
a drug delivery device ;
a sensor assembly comprising a sensor element configured to be arranged external to the drug delivery device, wherein the drug delivery device is configured to be held by a user of the drug delivery device through gripping of the sensor element, wherein the sensor element is configured to detect a contact area of a hand of the user when the user grips the sensor element; and
a communication link between the electronic device and the drug delivery device, wherein the electronic device is configured to receive the detected contact area via the communication link,
wherein the electronic device is configured to provide the detected contact area to an output unit.

33. The system of claim 32, wherein the drug delivery device is an injection device configured to inject a medicine for the treatment of a chronic disease.

34. The system of claim 32, wherein the sensor assembly is configured to be mounted to an outer surface of a body of the drug delivery device.

35. The system of claim 32, wherein the communication link comprises an electronic module configured to transmit data representing a detection result of the sensor element to the external electronic device.
